(19) United States
(12) Patent Application Publication
(10) Pub. No.: US 2026/0029274 A1
(43) Pub. Date: Jan. 29, 2026

(54) SYSTEMS AND METHODS FOR DETERMINING A SENSOR POWER RATIO

(71) Applicant: Covidien LP, Mansfield, MA (US)

(72) Inventors: Jacob D. Dove, Lafayette, CO (US); Linden A. Reustle, Milliken, CO (US); Scott James McGonigle, Loanhead (GB); Pedro M. Elias Monteiro Gomes, Sobral de Monte Agraco (PT)

(73) Assignee: Covidien LP, Mansfield, MA (US)

(21) Appl. No.: 19/251,616

(22) Filed: Jun. 26, 2025

Related U.S. Application Data
(60) Provisional application No. 63/675,798, filed on Jul. 26, 2024.

(51) Int. Cl. G01J 1/02; A61B 5/1455; A61B 5/1495; G01J 1/42

(52) U.S. Cl. CPC G01J 1/0295; A61B 5/1495; G01J 1/42; A61B 5/14551; G01J 2001/4252

(57) ABSTRACT

A calibration system includes a sensor including a light emitter to emit a first light and a second light, and a detector to detect the first light and the second light. The calibration system also includes a calibration structure, processing circuitry, and a memory including instructions that, when executed by the processing circuitry, cause the processing circuitry to instruct a first light-emitting diode (LED) of the light emitter to emit the first light at a first wavelength and a second LED of the light emitter to emit the second light at a second wavelength, receive a signal indicative of the first light at the first wavelength and the second light at the second wavelength, determine a first power at the first wavelength and a second power at the second wavelength based on the signal, and determine a power ratio as a ratio of the first power and the second power.

### CROSS-REFERENCE TO RELATED APPLICATION(S)

This application claims the benefit of priority to U.S. Provisional Application No. 63/675,798, filed Jul. 26, 2024, the entire disclosure of which is incorporated herein by reference in its entirety.

### TECHNICAL FIELD

The present disclosure generally relates to medical monitoring devices (e.g., sensors), and more specifically to systems and methods for determining a sensor power ratio and adjusting one or more detected signals based on the sensor power ratio.

### BACKGROUND

Various medical monitoring devices may be used to monitor physiological characteristics of an individual. For example, various sensors may be used to measure temperature, pressure, oxygen, and other physiological characteristics of the individual. One such sensor, a pulse oximetry sensor, may be used to measure oxygen saturation levels in blood of the individual by utilizing wavelengths of light. In this manner, the pulse oximetry sensor may provide physiological parameters related to respiratory and circulatory systems of the individual.

Variations in components of pulse oximetry sensors may affect light emission and detection, and thus affect pulse oximetry readings. Further, in certain cases, physiological factors, such as skin (e.g., tissue) pigmentation of a patient, skin thickness of the patient, and/or skin abnormalities (e.g., scarring) of the patient, may affect light absorption and scattering and lead to errors in pulse oximetry readings. For example, skin pigmentation may contribute to errors in a blood oxygen saturation (SpO₂) value.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it may be understood that these statements are to be read in this light, and not as admissions of prior art.

### SUMMARY

Certain embodiments commensurate in scope with the originally claimed subject matter are summarized below. These embodiments are not intended to limit the scope of the disclosure. Indeed, the present disclosure may encompass a variety of forms that may be similar to or different from the embodiments set forth below.

In certain embodiments, a method for determining a power ratio of a sensor at manufacturing, the method includes instructing, via a processor, a first light-emitting diode (LED) and a second LED of the sensor to emit light onto a calibration structure receiving, via the processor, a signal indicative of the light detected by a detector of the sensor, determining, via the processor and based on the signal, a first photocurrent generated by the detector due to a respective portion of the light emitted by the first LED and a second photocurrent generated by the detector due to a respective portion of the light emitted by the second LED, determining, via the processor, a first power for the first LED based on the first photocurrent and an attenuation factor of the calibration structure, determining, via the processor, a second power for the second LED based on the second photocurrent and the attenuation factor of the calibration structure, and determining, via the processor, the power ratio as a ratio of the first power and the second power.

In certain embodiments, a calibration system includes a sensor that includes a light emitter to emit a first light and a second light, and a detector to detect the first light and the second light. The calibration system also includes a calibration structure, calibration processing circuitry, and a calibration memory including instructions that, when executed by the calibration processing circuitry, cause the calibration processing circuitry to instruct a first light-emitting diode (LED) of the light emitter to emit the first light at a first wavelength and a second LED of the light emitter to emit the second light at a second wavelength, receive a signal indicative of the first light at the first wavelength and the second light at the second wavelength, determine a first power at the first wavelength and a second power at the second wavelength based on the signal, and determine a power ratio as a ratio of the first power and the second power.

Various refinements of the features noted above may exist in relation to various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. The brief summary presented above is intended only to familiarize the reader with certain aspects and context of embodiments of the present disclosure without limitation to the claimed subject matter.

### BRIEF DESCRIPTION OF THE DRAWINGS

Advantages of the disclosed techniques may become apparent upon reading the following detailed description and upon reference to the drawings in which:

FIG. 1 is a perspective view of an embodiment of a medical monitoring system configured to monitor oxygen saturation, in accordance with an aspect of the present disclosure;

FIG. 2 is a block diagram of an embodiment of the medical monitoring system of FIG. 1, in accordance with an aspect of the present disclosure;

FIG. 3 is a block diagram of a calibration system and a sensor, in accordance with an aspect of the present disclosure

FIG. 4 is a perspective view of a sensor and an integrating sphere as the calibration system of FIG. 3 employed at manufacturing to determine a power ratio, in accordance with an aspect of the present disclosure;

FIG. 5 is a flow diagram of a method of manufacturing to determine a power ratio of a sensor based on an integrating sphere, in accordance with an aspect of the present disclosure;

FIG. 6 is a perspective view of a sensor and a calibration block as the calibration system of FIG. 3 employed at manufacturing to determine a power ratio, in accordance with an aspect of the present disclosure;

FIG. 7 is a flow diagram of a method of manufacturing to determine an attenuation factor for a calibration block, in accordance with an aspect of the present disclosure;

FIG. 8 is a flow diagram of a method of manufacturing to determine a power ratio of a sensor based on a calibration block, in accordance with an aspect of the present disclosure; and

FIG. 9 is a flow diagram of a method for adjusting a detected signal, in accordance with an aspect of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

It is presently recognized that it may be desirable to account for variations in components of sensors in order to obtain accurate physiological measurements (e.g., oxygen saturation (SpO₂) values). For example, algorithms may account for the variations in the components of the sensors to generate accurate physiological measurements. Further, certain variations of the sensors may be utilized in other ways, such as to normalize certain inputs and/or features of signals generated by the sensors.

It is presently recognized that it may be desirable to identify and correct for a differential spectral loss due to various physiological factors of skin (e.g., skin pigmentation, skin thickness, skin abnormalities, skin damage) of a patient. Indeed, the physiological factors of the skin may result in different light absorption or scattering (e.g., within a spectral bandwidth of interest), which without embodiments disclosed herein, may result in error in an oxygen saturation (SpO₂) value determined and output by a monitor based on signals from the sensor. Certain existing systems may determine measurements, such as a direct current (DC) ratio (e.g., a ratio of DC components), and adjust the SpO₂ based on these measurements. However, it is presently recognized that the DC ratio may be influenced by light emission and/or efficiency of one or more light emitters of the sensor. Thus, it may be desirable to determine a power ratio of the sensor (e.g., at a manufacturing stage) and to normalize the DC ratio to remove the influence of the light emission and/or efficiency of the one or more light emitters when using the DC ratio to adjust the SpO₂.

Accordingly, the present disclosure generally relates to systems and methods for determining a power ratio (e.g., a ratio of a power of a red light emitting diode (LED) of a sensor to a power of an infrared (IR) LED of the sensor) of the sensor at a manufacturing stage of the sensor. A calibration system may be coupled to (e.g., in communication with, wired or wirelessly) the sensor 14. The calibration system may include a calibration structure, which may include an integrating sphere or a calibration block (e.g., polymer block, such as a polytetrafluoroethylene (PTFE) block; optical phantom, such as three-dimensional model that mimics tissue optical properties). In an embodiment, an operator, such as human operator and/or an actuator (e.g., actuated by any suitable controller), may place the red LED and the IR LED at a first aperture (e.g., hole, opening) of the calibration structure, such as the integrating sphere. The integrating sphere may include a hollow, spherical shell (e.g., enclosure) coated internally (e.g., on an interior surface) and/or externally (e.g., on an exterior surface) with a reflective material. A calibration system coupled to the sensor may instruct the red LED of the sensor to emit light at a first wavelength and the IR LED to emit light at a second wavelength into the integrating sphere at the aperture. Due to the reflective interior, the integrating sphere may cause the light (e.g., a total radiant flux) to be uniform in intensity and spatial distribution enabling an increase in accuracy of measurements.

The integrating sphere may include a second aperture, which includes or is coupled to a calibrated detector (e.g., separate from a detector of the sensor) that may detect the light at the first wavelength of the red LED and the light at the second wavelength of the IR LED. The calibration system may be coupled to the calibrated detector and may receive a signal (e.g., transmitted by the calibrated detector) indicative of the light detected by the calibrated detector. The calibration system may then determine a power of the red LED and a power of the IR LED based on the signal. Moreover, the calibration system may determine the power ratio by dividing the power of the red LED by the power of the IR LED (or vice versa) and transmit the power ratio to the sensor for storage in a memory of the sensor.

In another embodiment, the operator may place the sensor, which may include the red LED, the IR LED, and the detector, on the calibration structure, which may include the calibration block. The calibration block may be characterized (e.g., calibrated), such that an attenuation factor associated with the calibration structure is measured and recorded via the calibration system. The calibration system may then instruct the red LED to emit light at the first wavelength and the IR LED to emit light at the second wavelength. The detector may detect the light at the first wavelength of the red LED and the light at the second wavelength of the IR LED and generate a signal indicative of the light detected by the detector.

The calibration system may receive the signal indicative of the light detected by the detector after the light passes through the calibration block. The calibration system may determine a photocurrent associated with the red LED and a photocurrent associated with the IR LED based on the signal. Further, the calibration system may determine the power of the red LED and the power of the IR LED based on the photocurrent and an attenuation factor of the calibration block. Further, the calibration system may determine the power ratio by dividing the photocurrent of the red LED by the photocurrent of the IR LED (or vice versa) and transmit the power ratio to the sensor for storage in the memory of the sensor. In this manner, a monitor may receive the power ratio from the sensor and normalize detected signals (e.g., features of the signals, such as a DC ratio) based on the power ratio to account for variations of optical components, such as variations of the red LED and/or the IR LED.

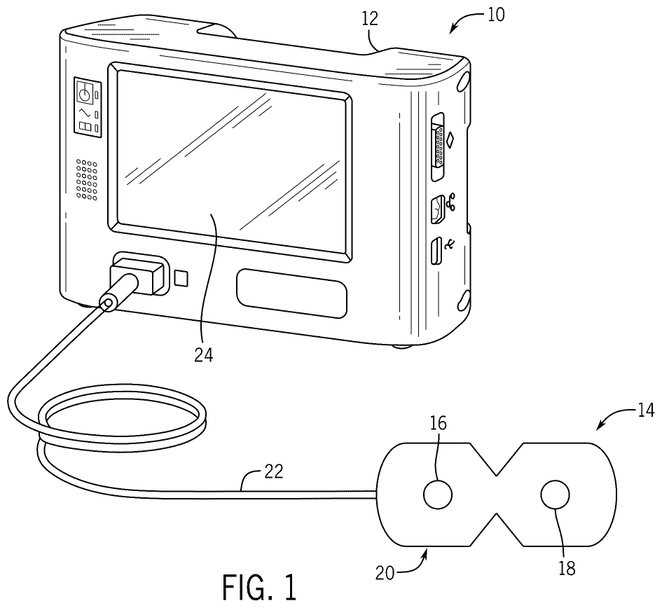

With the foregoing in mind, FIG. 1 is a perspective view of an embodiment of a medical monitoring system 10 that includes a patient monitor 12 (also referred to herein as “the monitor 12”) that may be used in conjunction with a medical sensor 14 (also referred to herein as “the sensor 14”). In the illustrated example, the monitor 12 is a pulse oximetry monitor and the sensor 14 is a pulse oximetry sensor. In such cases, the monitor 12 is configured to process photoplethysmography (PPG) signals to calculate oxygen saturation (SpO₂). It should be appreciated that the medical monitoring system 10 may be configured to obtain any of a variety of medical measurements and the techniques described herein may be adapted for use with any variety of monitors and sensors. By way of non-limiting example, in some embodiments, the monitor 12 may include a regional oximeter and the sensor 14 may include a regional saturation sensor. In such cases, the monitor 12 is configured to process the PPG signals to calculate regional oxygen saturation (rSO2). Additionally, although the depicted embodiments illustrate the sensor 14 configured for use on a patient's finger, it should be understood that the sensor 14 may be adapted for use at other tissue locations, such as a forehead, temple, earlobe, toe, foot, heel, ankle, stomach, chest, back, neck, wrist, thigh, or any other suitable measurement site.

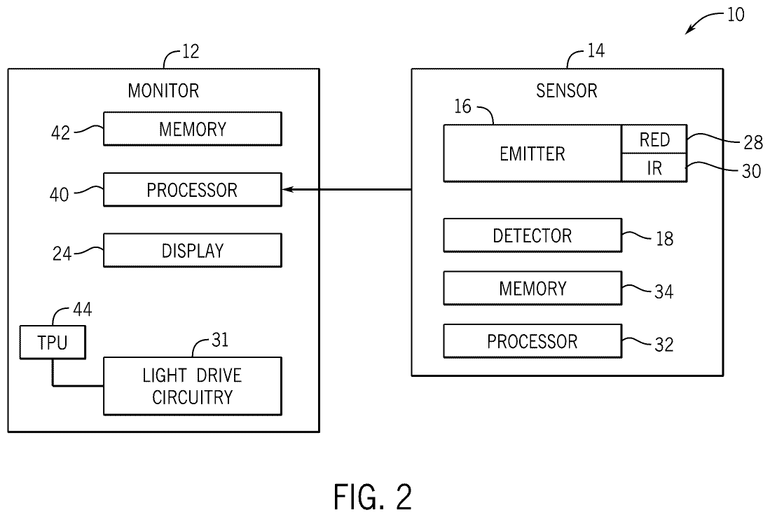

In FIG. 1, the sensor 14 includes one or more emitters 16 (collectively referred to herein as “the emitter 16” for convenience) and one or more detectors 18 (collectively referred to herein as “the detector 18” for convenience). The emitter 16 emits wavelengths of light that passes through blood perfused tissue, and the detector 18 detects the light as reflected or transmitted by the tissue. Additional details regarding the emitter 16 and the detector 18 will be described below with respect to FIG. 2. In certain embodiments, the sensor 14 may include sensing components in addition to the emitter 16 and the detector 18. The sensor 14 includes a sensor body 20 that may include multiple layers, such as a backing, adhesives, and so on. The sensor body 20 may also include or support a flexible circuit with various components. In certain embodiments, the medical monitoring system 10 may include multiple sensors 14 at multiple locations.

The sensor 14 is communicatively coupled to the monitor 12. In the illustrated embodiment, the sensor 14 is coupled to the monitor 12 via a cable 22. The cable 22 may interface directly with the sensor 14 and may include multiple conductors (e.g., wires) to transmit signals and/or receive signals. Additionally or alternatively, the sensor 14 may communicate with the monitor 12 wirelessly (e.g., the sensor 14 and the monitor 12 include wireless transceivers configured to communicate via any suitable wireless protocol). For example, the sensor 14 may include a transceiver that enables wireless signals to be transmitted to and/or received from an external device (e.g., the monitor 12). Additionally, the multiple conductors or the transceiver may transmit a raw digitized detector signal, a processed digitized detector signal, or a calculated physiological parameter, as well as any data (e.g., sensor identifier data, neural network data, coefficient data, power transfer unit (PTU) characterization data, power ratio data) that may be stored in the sensor 14.

In operation, the monitor 12 may receive a signal from the sensor 14, and the monitor 12 may be configured to calculate or measure one or more physiological parameters based on the signal. In particular, the monitor 12 may include a processor configured to execute code (e.g., stored in a memory of the monitor 12 or received from another device) for filtering and processing the signal from the sensor 14 to calculate physiological parameters, such as oxygen saturation. The monitor 12 may additionally or alternatively calculate any variety of physiological parameters, such as arterial blood oxygen saturation, regional or tissue oxygen saturation, pulse rate, respiration rate, blood pressure, blood pressure characteristic measure, autoregulation status, brain activity, temperature, or any other suitable physiological parameter.

Additionally, as illustrated in FIG. 1, the monitor 12 includes a display 24 configured to display one or more calculated physiological parameters, such as the oxygen saturation (e.g., adjusted or corrected based on a power ratio of the sensor; adjusted or corrected based on a DC ratio normalized based on a power ratio of the sensor 14). The display 24 may also display other information, such as instructions to charge the sensor 14, alarm indications, settings, and so forth. In certain embodiments, the display 24 may be a touch screen display. The monitor 12 may include various input components, such as the touch screen display, knobs, switches, keys and keypads, buttons, and so forth, to provide for operation and configuration of the monitor 12. The monitor 12 may also include one or more indicator lights and one or more speakers. The monitor 12 may also include additional slot(s) or wireless interfaces (e.g., channels) to connect to additional devices, such as additional sensors to monitor additional physiological parameters of the patient and/or to monitor physiological parameters of other patients at one time.

Furthermore, one or more functions of the monitor 12 disclosed herein may also be implemented directly in the sensor 14, or by any other suitable device. For example, in some embodiments, the sensor 14 may include one or more processing components configured to calculate physiological parameters, such as oxygen saturation. The sensor 14 may have varying levels of processing power, and may output data in various stages to the monitor 12. For example, in some embodiments, the data output to the monitor 12 may be analog signals, such as detected light signals (e.g., pulse oximetry signals or regional saturation signals), or processed data.

Further, in some embodiments, the sensor 14 may include a battery to provide power to components of the sensor 14. For example, the sensor 14 may be configured to operate in a wireless mode and, at times, may not receive power from the monitor 12 while operating in the wireless mode. In some embodiments, the battery may be a rechargeable battery such as, for example, a lithium ion, a lithium polymer, a nickel-metal hydride, a nickel-cadmium battery, or any other suitable rechargeable battery. In other embodiments, any suitable power source may be utilized, such as, one or more capacitors or an energy harvesting power supply (e.g., a motion generated energy harvesting device, thermoelectric generated energy harvesting device, or any other suitable energy harvesting power supply).

Turning to FIG. 2, a simplified block diagram of the medical monitoring system 10 is illustrated in accordance with an embodiment. As described herein, the sensor 14 includes the emitter 16 and the detector 18. The emitter 16 includes two light emitting diodes (LEDs) that are configured to emit at least two wavelengths of light, e.g., a red LED 28 configured to emit wavelengths of light within the red spectrum and an infrared (IR) LED 30 configured to emit wavelengths of light within the infrared or near infrared spectrum. In one embodiment, the LEDs 28, 30 emit light in a range of about 600 nanometers (nm) to about 1000 nm. In one embodiment, the red LED 28 is configured to emit light between approximately 600 nm and 735 nm, and the IR LED 30 is configured to emit light between approximately 800 nm and 1000 nm. It should be noted that the emitter 16 may also transmit 3, 4, or 5 or more wavelengths of light in any suitable application.

As discussed in more detail herein, a light drive circuitry 31 of the monitor 12 may provide respective drive currents to the LEDs 28, 30 to cause the LEDs 28, 30 to emit respective wavelengths of light. It should be understood that, as used herein, the term “light” may refer to one or more of ultrasound, radio, microwave, millimeter wave, infrared, near-infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

The emitter 16 emits light that passes through blood perfused tissue, and the detector 18 detects the light as reflected or transmitted by the tissue. The emitter 16 and the detector 18 may be arranged in a transmission configuration or a reflectance configuration with respect to one another. In the transmission configuration, the light enters the detector 18 after passing through the tissue of the patient. In the reflectance configuration, the light is reflected by elements in the tissue of the patient to enter the detector 18. In any case, the detector 18 may generate a signal (e.g., PPG signal) indicative of an intensity of the light received at the detector 18, and the detector 18 may send the signal to the monitor 12.

A signal representing light intensity versus time or a mathematical manipulation of this signal (e.g., a scaled version thereof, a log taken thereof, a scaled version of a log taken thereof) may be referred to as the PPG signal. Additionally, the term “PPG signal,” as used herein, may also refer to an absorption signal (e.g., representing an amount of light absorbed by the tissue) or any suitable mathematical manipulation thereof. The amount of light detected or absorbed may then be used to calculate any of a number of physiological parameters, including oxygen saturation (e.g., the saturation of oxygen in pulsatile blood, SpO2), an amount of a blood constituent (e.g., oxyhemoglobin), and/or a physiological rate (e.g., pulse rate or respiration rate; when each individual pulse or breath occurs). For SpO2, red and infrared (IR) wavelengths may be used because it has been observed that highly oxygenated blood will absorb relatively less Red light and more IR light than blood with a lower oxygen saturation. By comparing the intensities of two wavelengths at different points in the pulse cycle, it is possible to estimate the blood oxygen saturation of hemoglobin in arterial blood, such as from empirical data that may be indexed by values of a ratio, a lookup table, from curve fitting, or other interpolative techniques.

As shown, the sensor 14 also includes one or more processors 32 (collectively referred to herein as a processor for convenience) and one or more memory devices 34 (collectively referred to herein as a memory for convenience). The memory 34 may include or be an add-only memory, a rewriteable integrated circuit, or other suitable memory type. The processor 32 may include a processing system or processing circuitry to perform operations and execute instructions stored in the memory 34. Further, at least a portion of the memory 34 is erasable and reprogrammable. For example, the memory 34 may include an erasable programmable read-only memory (EPROM) (e.g., an electrically erasable programmable read-only memory (EEPROM)), which is erasable and reprogrammable, and thus enables a user to provide inputs to write data to it. In some embodiments, the memory 34, such as the EPROM, may enable storage of the power ratio (e.g., a ratio of the power of the red LED 28 to the power of the IR LED 30) of the sensor 14, which may be determined at manufacturing. The memory 34 also stores other information about the sensor 14, such as a type of sensor, calibration information, a power of the red LED 28, a power of the IR LED 30, and so forth.

When accessed by the monitor 12, the information about the sensor 14 may enable the monitor 12 to calculate oxygen saturation and/or other physiological parameters using the signal received from the detector 18. Further, when accessed by the monitor 12, the power of the red LED 28, the power of the IR LED 30, and/or the power ratio may enable the monitor 12 to normalize (e.g., adjust) the signal received (e.g., and/or features of the signal, such as the DC ratio of the signal). In certain embodiments, the sensor 14 may include sensing components in addition to the emitter 16 and the detector 18. For example, in one embodiment, the sensor 14 may include one or more actively powered electrodes (e.g., four electrodes) to obtain an electroencephalography signal.

As shown, the monitor 12 includes one or more processors 40 (collectively referred to herein as a processor for convenience), a memory 42, and the display 24. The processor 40 may include a processing system or processing circuitry to perform operations and execute instructions stored in the memory 42. The processor 40 may process the signal received from the detector 18, such as by performing synchronized demodulation, amplification, and filtering of the signal. The processor 40 may process the signal received from the detector 18 to calculate one or more physiological parameters, such as the oxygen saturation, using various algorithms. Coefficients utilized in the algorithms may be accessed by the processor 40 from the memory 34 or determined by the processor 40 based on the calibration information of the sensor 14, for example.

As shown, the monitor 12 includes a time processing unit (TPU) 44, which may be controlled by the processor 40 and is configured to provide timing control signals to the light drive circuitry 31 and optionally to other parts of the medical monitoring system 10. The light drive circuitry 31 may control when the red LED 28 and the IR LED 30 are illuminated and/or a drive current provided to the red LED 28 and the IR LED 30. It should be appreciated that one or more functions or components of the monitor 12 disclosed herein may also be implemented directly in the sensor 14, or by any other suitable device.

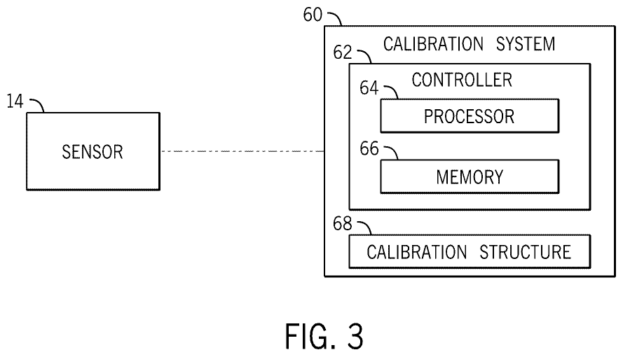

FIG. 3 is a block diagram of a calibration system 60 and the sensor 14, in accordance with an aspect of the present disclosure. Indeed, the sensor 14 may be communicatively coupled (e.g., wired or wirelessly) to the calibration system 60. The calibration system 60 includes a controller 62 (e.g., electronic controller) that includes one or more processors 64 (collectively referred to herein as a processor for convenience) and a memory 66. The calibration system 60 also includes a calibration structure 68, which may either include an integrating sphere or a calibration structure (e.g., calibration block, polymer block, polytetrafluoroethylene (PTFE) block; optical phantom).

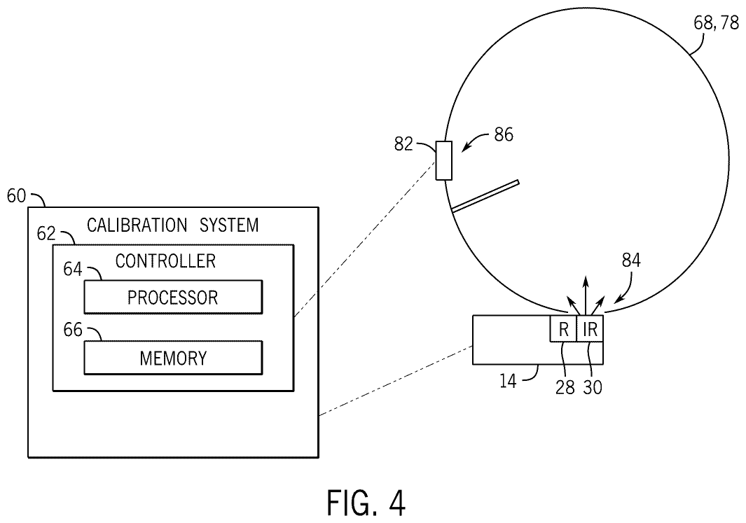

The processor 64 may include a processing system or processing circuitry to perform operations and execute instructions stored in the memory 66. For example, the processor 64 may instruct the red LED 28 and/or the IR LED 30 of the sensor 14 (see FIG. 2) to emit light, receive signals indicative of the light (e.g., detected by the detector 18 of the sensor 14 (see FIG. 2) or a separate calibrated detector), determine a power associated with the red LED 28 and the IR LED 30, determine the power ratio of the red LED 28 to the IR LED 30, and so on. In some embodiments, the calibration system 60 may be coupled to and receive signals from a calibrated detector (e.g., separate from the detector 18 of the sensor 14). Additional details regarding operations performed by the calibration system 60 will be described below with respect to FIGS. 4-8.

With the foregoing in mind, FIG. 4 is a perspective view of the sensor 14, the calibration system 60, and a calibrated detector 82 (or a spectrophotometer) employed in manufacturing to determine a power ratio, in accordance with an aspect of the present disclosure. As illustrated, the calibration structure 68 may include or have a form of an integrating sphere 78. An operator, such as a human operator or an actuator (e.g., instructed to actuate via the calibration system 60), may place the red LED 28 and the IR LED 30 at a first aperture 84 (e.g., hole, opening) of the integrating sphere 78. The integrating sphere 78 may include a hollow, spherical shell (e.g., enclosure, cavity) coated internally (e.g., on an interior surface) and/or externally (e.g., on an exterior surface) with a reflective material. For example, the reflective material may include a diffuse reflective coating (e.g., barium sulfate), a metallic coating (e.g., aluminum), or the like. The reflective material of the integrating sphere 78 may enable scattering and reflecting of light, resulting in a uniform distribution of light throughout the integrating sphere.

After placement of the sensor 14, the calibration system 60 may instruct the red LED 28 to emit light at a first wavelength and the IR LED 30 to emit light at a second wavelength. Thus, the light at the first wavelength and the light at the second wavelength may enter the integrating sphere 78 through the positioning of the red LED 28 and the IR LED 30 at the first aperture 84. The integrating sphere 78 may also include a second aperture 86, and the calibrated detector 82 may be positioned at the second aperture 86. It should be noted that the calibrated detector 82 is separate (e.g., distinct) from the detector 18 of the sensor 14 (see FIG. 2). As an example, the calibrated detector 82 may be calibrated based on one or more reference values (e.g., known standards). Further, the calibrated detector 82 may be utilized as a reference instrument to measure, verify, and/or enable maintenance of accuracy of light intensity measurements at the integrating sphere 78. In this manner, the calibrated detector 82 may detect the light at the first wavelength of the red LED 28 and the light at the second wavelength of the IR LED 30 and generate a signal indicative of the light at the first wavelength and the light at the second wavelength. The calibrated detector 82 may then transmit the signal indicative of the light to the calibration system 60.

The calibration system 60 may receive the signal indicative of the light and determine a first power (e.g., a first intensity) at the first wavelength emitted by the red LED 28 and a second power (e.g., a second intensity) at the second wavelength emitted by the IR LED 30 based on the signal. Indeed, the calibration system 60 may apply one or more filters (e.g., optical filters) to the signal to separate (e.g., isolate) the light at the first wavelength and the light at the second wavelength. Further, the calibration system 60 may measure and/or determine the first power at the first wavelength and the second power at the second wavelength. The calibration system 60 may then determine a power ratio of the sensor 14 by dividing the first power by the second power and transmit the power ratio to the sensor 14 for storage in the memory 34 of the sensor 14.

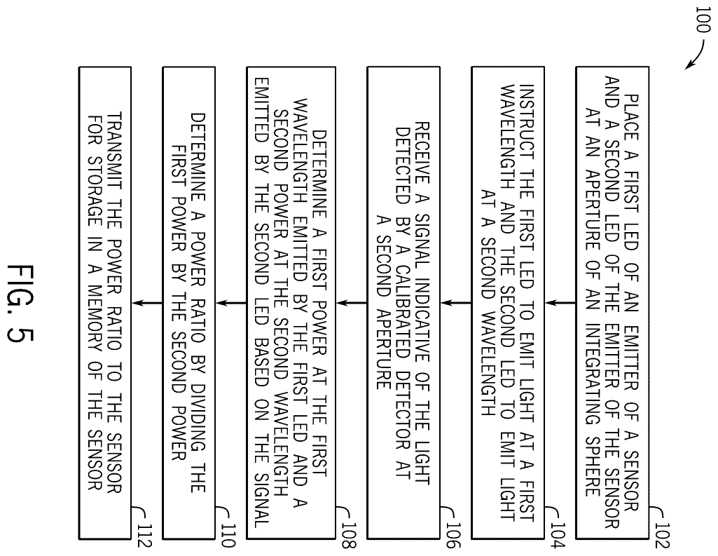

FIG. 5 is a flow diagram of a method of manufacturing 100 to determine a power ratio of a sensor based on an integrating sphere, in accordance with an aspect of the present disclosure. It should be noted that at least some steps of the method 100 may be performed as an automated procedure by a system, such as the calibration system 60 of FIG. 3. Further, certain steps or portions of the method 100 may be performed by certain devices, such as the monitor 12 (e.g., the processing system that includes the processor 40) and/or the sensor 14 (e.g., the processing system that includes the processor 32). In addition, certain steps or portions of the method 100 may be performed by an operator, such as human operator and/or an actuator (e.g., actuated by any suitable controller, such as the controller 62). It should be appreciated that “the processing system” as used herein may refer to components (e.g., the processor 40, the processor 32, the processor 64) in the monitor 12, the sensor 14, and/or the calibration system 60. Although the flow diagram illustrates the steps in a certain sequence, it should be understood that the steps may be performed in any suitable order and certain steps may be carried out simultaneously, where appropriate.

At block 102, an operator, such as a human operator or an actuator, places a first LED (e.g., a red LED) of an emitter of a sensor and a second LED (e.g., an IR LED) of the emitter of the sensor at a first aperture of an integrating sphere. As described herein, the integrating sphere includes a spherical enclosure with a reflective inner surface, enabling scattering and an even distribution of light from any direction. At block 104, a controller of the calibration system instructs the first LED to emit light at a first wavelength and the second LED to emit light at a second wavelength (e.g., via a particular drive current (e.g., 20 milliamperes (mA)). For example, the first wavelength and/or the second wavelength may be at a range between 600 nm to 1000 nm. The light at the first wavelength and the light at the second wavelength enters the integrating sphere by way of the first LED placement and the second LED placement at the first aperture of the integrating sphere. The integrating sphere then scatters and evenly distributes the light within the integrating sphere (e.g., simulating a uniform tissue environment).

At block 106, the controller of the calibration system receives a signal indicative of the light detected by a calibrated detector at a second aperture of the integrating sphere. Indeed, the calibrated detector may measure an amount of light, light intensity, wavelength distribution, and/or spectral distribution of the light within the integrated sphere and generate a signal indicative of the measurements of the light to provide to the controller of the calibration system. At block 108, the controller of the calibration system determines a first power (e.g., a first total radiant flux) at the first wavelength emitted by the first LED and a second power (e.g., a second total radiant flux) at the second wavelength emitted by the second LED based on the signal. Indeed, the controller of the calibration system processes the signal to interpret the first power and the second power.

At block 110, the controller of the calibration system determines a power ratio by dividing the first power by the second power. Further, at block 112, the controller of the calibration system transmits (e.g., provides) the power ratio to the sensor for storage in a memory of the sensor. In some embodiments, the controller of the calibration system may additionally or alternatively transmit the first power and the second power to the memory of the sensor. For example, the memory may include the EPROM, and the first power, the second power, and/or the power ratio may be stored on the EPROM of the sensor. In this manner, a monitor (or any other suitable sensor electronics) may read the first power, the second power, and/or the power ratio and adjust one or more detected signals based on the first power, the second power, and/or the power ratio to account for variations in the optical components (e.g., the first LED, the second LED).

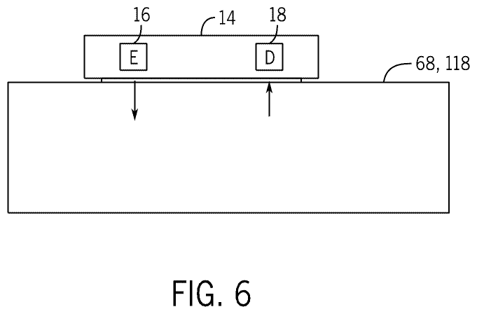

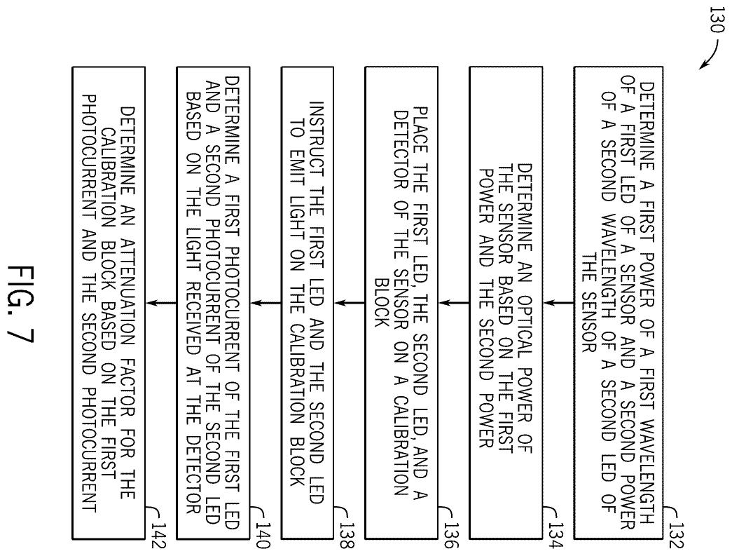

As described herein, in some embodiments, the calibration structure may include a calibration block (e.g., polymer block, polytetrafluoroethylene (PTFE) block; optical phantom), which may enable a determination of the power ratio of the sensor 14. FIG. 6 is a perspective view of the sensor 14 and the calibration system 60 employed at manufacturing to determine the power ratio, in accordance with an embodiment of the present disclosure. As illustrated, the calibration structure 68 may include the calibration block 118. The calibration block 118 may include physical properties that enable verification of performance of medical monitoring devices (e.g., the sensor 14). Moreover, a material composition of the calibration block 118 may be selected based on its ability to mimic properties of a substance being studied or measured. For example, the material composition may include materials that simulate tissue of a patient. As will be described in further detail below with respect to FIG. 7, the calibration block 118 may be characterized to be used in manufacturing.

For the calibration block 118, certain factors, such as an attenuation factor, may determine how light interacts with and/or travels through the calibration block 118. Thus, before determining the power ratio, the calibration system 60 may determine an attenuation factor of the calibration block 118. For example, the calibration system 60 may utilize a first sensor 14 (e.g., a test sensor) with a known power of its red LED 28 (see FIG. 2) and a known power of its IR LED 30 (see FIG. 2) to determine the attenuation factor of the calibration block 118. Furthermore, once the attenuation factor of the calibration block 118 is known, the operator may place the red LED 28, the IR LED 30, and the detector 18 of the sensor 14 on the calibration block 118. After placement, the calibration system 60 may instruct the red LED 28 to emit the light at the first wavelength and the IR LED 30 to emit the light at the second wavelength. Further, the calibration system 60 may provide a particular drive current (e.g., 20 mA) to drive the red LED 28 and the IR LED 30 to emit light.

The detector 18 detects the light at the first wavelength and the light at the second wavelength reflected or transmitted by the calibration block 118 and generates a signal indicative of the light at the first wavelength and the light at the second wavelength. Indeed, the light at the first wavelength and the light at the second wavelength may enter the detector 18 after passing through the calibration block 118. The calibration system 60 may then determine a first photocurrent associated with the red LED 28 and a second photocurrent associated with the IR LED 30 based on the light at the detector 18. Further, the calibration system 60 may then determine a first power of the red LED 28 based on the first photocurrent and the attenuation factor of the calibration block 118, as well as a second power of the IR LED 30 based on the second photocurrent and the attenuation factor of the calibration block 118. Further, the calibration system 60 may determine the power ratio by dividing the first power of the red LED 28 by the second power of the IR LED 30 (or vice versa). Advantageously, the calibration block 118 enables determining the power ratio using the detector 18 of the sensor 14, as well as other components (e.g., bandaging) of the sensor 14 that may affect power. Accordingly, the calibration block 118 enables determining the power ratio for the sensor 14 as a whole (e.g., including the detector 18 and the bandaging that will be be applied to the patient when the sensor 14 is used in a clinical setting), and the power ratio for the sensor as a whole may also be referred to herein as a power transfer unit (PTU) for the sensor 14.

With the foregoing in mind, FIG. 7 is a flow diagram of a method of manufacturing 130 to determine an attenuation factor (e.g., an attenuation coefficient) for a calibration block, in accordance with an aspect of the present disclosure. It should be noted that at least some steps of the method 130 may be performed as an automated procedure by a system, such as the calibration system 60 of FIG. 3. Further, certain steps or portions of the method 130 may be performed by certain devices, such as the monitor 12 (e.g., the processing system that includes the processor 40) and/or the sensor 14 (e.g., the processing system that includes the processor 32). In addition, certain steps of portions of the method 130 may be performed by an operator such as human operator and/or an actuator (e.g., actuated by any suitable controller, such as the controller 62). It should be appreciated that “the processing system” as used herein may refer to components (e.g., the processor 40, the processor 32, the processor 64) in the monitor 12, the sensor 14, and/or the calibration system 60. Although the flow diagram illustrates the steps in a certain sequence, it should be understood that the steps may be performed in any suitable order and certain steps may be carried out simultaneously, where appropriate.

At block 132, a controller of the calibration system determines a first power of a first wavelength of a first LED of a sensor (e.g., first sensor; test sensor) and a second power of a second wavelength of a second LED of the sensor (e.g., prior to the sensor placement onto the calibration block). For example, the controller of the calibration system may determine the first power and the second power by employing the method 100 described herein with respect to FIG. 5. Additionally, it should be noted that the first power of the first LED and the second power of the second LED are based on light emission at a set amount of drive current (e.g., a particular drive current; 20 mA).

At block 134, the controller of the calibration system determines an optical power of the sensor based on the first power and the second power. The optical power may include an amount of light power, such as the light intensity, that each LED of the sensor emits at the set amount of drive current. In some embodiments, the controller of the calibration system may also determine a responsivity of a detector of the sensor by measuring an output signal of the detector in response to a known input optical power. In this manner, the controller of the calibration system may record measurements of the optical power and the known responsivity of the detector, which may provide a baseline reference for an initial intensity of the light before interaction with the calibration block.

At block 136, an operator may place the first LED, the second LED, and the detector of the sensor on the calibration block. As described herein, the calibration block may include the material composition that mimics the tissue of the patient. At block 138, the controller of the calibration system instructs the first LED and the second LED to emit light on the calibration block. Indeed, the controller of the calibration system may instruct the first LED and the second LED to emit light at the set amount of drive current, such as the set amount of drive current described herein with respect to block 132.

Moreover, at block 140, the calibration system determines a first photocurrent of the first LED (e.g., associated with the first LED; due to respective light emitted by the first LED and detected at the detector after passing through the calibration block) and a second photocurrent of the second LED (e.g., associated with the second LED; due to respective light emitted by the second LED and detected at the detector after passing through the calibration block) received at the detector. That is, the detector captures (e.g., receives) the light emitted by the first LED and the light emitted by the second LED that has passed through and/or been reflected by the calibration block and provides a signal indicative of the light to the controller of the calibration system. The first photocurrent may include a measurement (e.g., in amperes per watt) of an amount of light emitted by the first LED that passes through the calibration block and reaches the detector. Moreover, the second photocurrent may include a measurement of an amount of light emitted by the second LED that passes through the calibration block and reaches the detector.

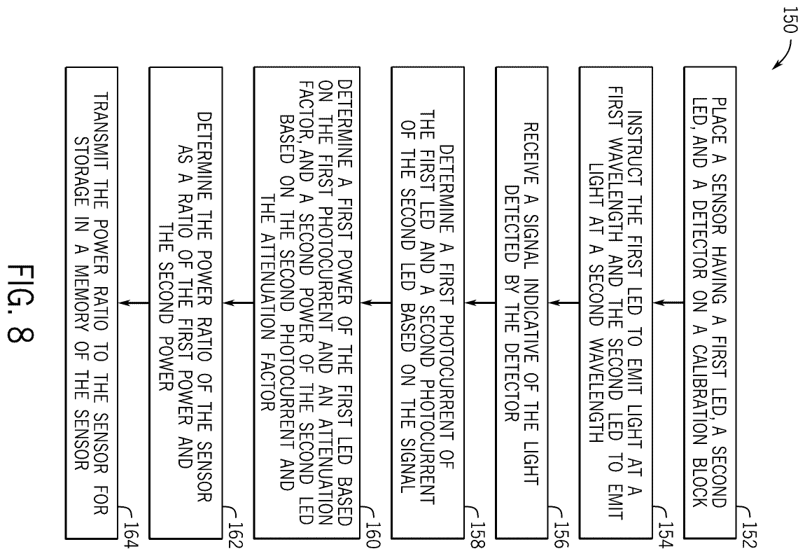

At block 142, the controller of the calibration system determines an attenuation factor for the calibration block based on the first photocurrent and the second photocurrent. That is, the first photocurrent and the second photocurrent may enable the controller of the calibration system to determine a change in the optical power of each LED of the sensor after placement of the sensor on the calibration block. Thus, the controller of the calibration system may determine the attenuation factor for the calibration block based on the change in the optical power. As such, the method 130 may enable the characterization of the calibration block, which may then enable the calibration system to efficiently and accurately determine respective power ratios for other sensors during manufacturing when employing the calibration block. It should be noted that the first photocurrent and the second photocurrent may enable the calibration system to determine the optical power of each of the first LED and the second LED. Indeed, the first photocurrent and the second photocurrent may provide a first measure of optical power (e.g., optical efficiency) of the first LED and a second measure of optical power (e.g., optical efficiency) of the second LED. It should be appreciated that the method 150 of FIG. 8 may be repeated for multiple sensors, such as during an assembly and/or calibration process that is part of the manufacturing process, to efficiently determine and store a respective power ratio for each of the multiple sensors.

With the foregoing in mind, FIG. 8 is a flow diagram of a method of manufacturing 150 to determine a power ratio (e.g., power transfer unit (PTU) characterization) of a sensor based on a calibration block, in accordance with an aspect of the present disclosure. It should be noted that at least some steps of the method 150 may be performed as an automated procedure by a system, such as the calibration system 60 of FIG. 3. Further, certain steps or portions of the method 150 may be performed by certain devices, such as the monitor 12 (e.g., the processing system that includes the processor 40) and/or the sensor 14 (e.g., the processing system that includes the processor 32). In addition, certain steps of portions of the method 150 may be performed by an operator such as human operator and/or an actuator (e.g., actuated by any suitable controller, such as the controller 62). It should be appreciated that “the processing system” as used herein may refer to components (e.g., the processor 40, the processor 32, the processor 64) in the monitor 12, the sensor 14, and/or the calibration system 60. Although the flow diagram illustrates the steps in a certain sequence, it should be understood that the steps may be performed in any suitable order and certain steps may be carried out simultaneously, where appropriate.

At block 152, an operator places the sensor having a first LED, a second LED, and a detector on a calibration block. Moreover, at block 154, a controller of the calibration system instructs the first LED to emit light at a first wavelength and the second LED to emit light at the second wavelength (e.g., via a particular drive current (e.g., 20 milliamperes (mA)). The detector of the sensor detects the light at the first wavelength and the light at the second wavelength that passes through the calibration block and generates a signal indicative of the light at the first wavelength and the light at the second wavelength. Therefore, at block 156, the controller of the calibration system receives the signal indicative of the light detected by the detector.

At block 158, the controller of the calibration system determines a first photocurrent of the first LED (e.g., associated with the first LED; due to respective light emitted by the first LED and detected at the detector after passing through the calibration block) and a second photocurrent of the second LED (e.g., associated with the second LED; due to respective light emitted by the second LED and detected at the detector after passing through the calibration block) based on the signal (e.g., indicative of the light received at the detector). At block 160, the controller of the calibration system determines a first power of the first LED based on the first photocurrent and an attenuation factor (e.g., such as the attenuation factor described herein with respect to FIG. 7), and a second power of the second LED based on the second photocurrent and the attenuation factor. At block 162, the calibration system determines the power ratio of the sensor as a ratio of the first power and the second power (e.g., by dividing the first power by the second power, or vice versa). Moreover, at block 164, the controller of the calibration system transmits (e.g., provides) the power ratio to the sensor for storage in a memory (e.g., an EPROM) of the sensor. In some embodiments, the controller of the calibration system may also transmit the first measure of optical efficiency (e.g., power, such as for the particular drive current) of the first LED and the second measure of optical efficiency (e.g., power, such as for the particular drive current) of the second LED. In this manner, a monitor (or any other suitable sensor electronics) may read the first optical efficiency (e.g., the power), the second optical efficiency (e.g., the power), and/or the power ratio and adjust one or more detected signals (e.g., features of the signals, such as a DC ratio) based on the first optical efficiency, the second optical efficiency, and/or the power ratio to account for variations in the optical components (e.g., the first LED, the second LED; variations in the optical components across multiple sensors).

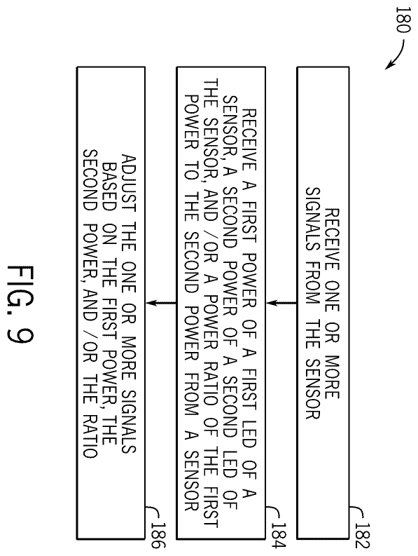

FIG. 9 is a flow diagram of a method 180 for adjusting a detected signal, in accordance with an aspect of the present disclosure. The method 180 disclosed herein includes various steps represented by blocks. It should be noted that at least some steps of the method 180 may be performed as an automated procedure by a system, such as the medical monitoring system 10 of FIG. 1. Further, certain steps or portions of the method 180 may be performed by certain devices, such as the monitor 12 (e.g., the processor 40) and/or the sensor 14 (e.g., the processor 32). Although the flow chart illustrates the steps in a certain sequence, it should be understood that the steps may be performed in any suitable order and certain steps may be carried out simultaneously, where appropriate.

At block 182, a processor of the monitor receives one or more signals from the sensor. For example, the one or more signals may be associated with a physiological parameter of a patient, such as a blood oxygen saturation level, an amount of light received from a first LED of the sensor, an amount of light received from a second LED of the sensor, and/or the like. The processor of the monitor may use the one or more input signals to determine (e.g., measure) a ratio of direct current (DC) components, which may include a ratio of the received light from the first LED (e.g., received red light) to received light from the second LED (e.g., received IR light). The ratio of DC components may enable the monitor to determine a correction factor, which may be utilized to determine (e.g., correct, adjust) an oxygen saturation for the patient. However, as described herein, light emission and/or efficiency of the first LED and the second LED may affect the ratio of DC components. That is, if the first LED and/or the second LED are more or less efficient, such that more or less light is emitted at a particular drive current, then the ratio of DC components may be affected.

Therefore, at block 184, the processor of the monitor receives a first power of the first LED of the sensor, a second power of the second LED of the sensor, and/or a power ratio of the first power to the second power from the sensor. Additionally, at block 186, the processor of the monitor adjusts (e.g., normalizes) the one or more signals (e.g., the ratio of DC components derived from the signals) received from the sensor based on the first power, the second power, and/or the power ratio. For example, the processor of the monitor may determine, based on the received values, that the second LED was more efficient and produced more light. Thus, the processor of the monitor may adjust the one or more signals based on the determined optical variation when determining the ratio of DC components.

In this manner, the processor of the monitor may account for variations in the optical components (e.g., by calibrating for the optical components of the sensor), such as the first LED and/or the second LED, when determining the ratio of DC components to be used in determining (e.g., correcting, adjusting) the blood oxygen saturation level. As such, embodiments described herein improve DC measurements recorded on tissue (e.g., skin) by removing an influence of the optical variations and/or sensor use by using measurements (e.g., the power ratio) determined during manufacturing. Accordingly, embodiments described herein also improve measurements and/or correction of the blood oxygen saturation level by enabling increased accuracy in determining the ratio of DC components.

It should be noted that although the method 180 is described as being performed by the monitor, in some embodiments, certain steps or portions of the method 180 may be performed by the sensor. For example, the sensor may perform blocks 184 and 186 and then transmit the adjusted one or more signals to the monitor for the monitor to display.

Further, it should be noted that while some of the techniques described herein are described as being performed by the calibration system at manufacturing, the techniques could be performed (e.g., to determine the power ratio) and/or repeated (e.g., for verification of the power ratio) at other times (e.g., in clinical settings). In some such cases, at least some portions of these techniques may be performed by a calibration system at the other times and/or by another system (e.g., the patient monitor 12 of FIG. 2).

In certain embodiments, a method for determining a power ratio of a sensor at manufacturing, the method includes instructing, via a processor, a first light-emitting diode (LED) and a second LED of the sensor to emit light onto a calibration structure receiving, via the processor, a signal indicative of the light detected by a detector of the sensor, determining, via the processor and based on the signal, a first photocurrent generated by the detector due to a respective portion of the light emitted by the first LED and a second photocurrent generated by the detector due to a respective portion of the light emitted by the second LED, determining, via the processor, a first power for the first LED based on the first photocurrent and an attenuation factor of the calibration structure, determining, via the processor, a second power for the second LED based on the second photocurrent and the attenuation factor of the calibration structure, and determining, via the processor, the power ratio as a ratio of the first power and the second power.

In certain embodiments, a calibration system includes a sensor that includes a light emitter to emit a first light and a second light, and a detector to detect the first light and the second light. The calibration system also includes a calibration structure, calibration processing circuitry, and a calibration memory including instructions that, when executed by the calibration processing circuitry, cause the calibration processing circuitry to instruct a first light-emitting diode (LED) of the light emitter to emit the first light at a first wavelength and a second LED of the light emitter to emit the second light at a second wavelength, receive a signal indicative of the first light at the first wavelength and the second light at the second wavelength, determine a first power at the first wavelength and a second power at the second wavelength based on the signal, and determine a power ratio as a ratio of the first power and the second power.

In certain embodiments, a medical monitoring system includes a sensor and a monitor. The sensor includes a sensor memory that stores a power ratio, wherein the power ratio is determined using a calibration structure, and wherein the calibration structure is an optical phantom with an attenuation coefficient. The monitor includes a port to communicatively couple to the sensor to receive a sensor signal and the power ratio from the sensor. The monitor also includes monitor processing circuitry to adjust the sensor signal based on the power ratio.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

1. A method for determining a power ratio of a sensor at manufacturing, the method comprising:
instructing, via a processor, a first light-emitting diode (LED) and a second LED of the sensor to emit light onto a calibration structure;
receiving, via the processor, a signal indicative of the light detected by a detector of the sensor;
determining, via the processor and based on the signal, a first photocurrent generated by the detector due to a respective portion of the light emitted by the first LED and a second photocurrent generated by the detector due to a respective portion of the light emitted by the second LED;
determining, via the processor, a first power for the first LED based on the first photocurrent and an attenuation factor of the calibration structure;
determining, via the processor, a second power for the second LED based on the second photocurrent and the attenuation factor of the calibration structure; and
determining, via the processor, the power ratio as a ratio of the first power and the second power.

2. The method of claim 1, wherein the calibration structure comprises a calibration block that forms an optical phantom.

3. The method of claim 2, wherein the calibration block comprises a polymer material.

4. The method of claim 3, wherein the polymer material comprises polytetrafluoroethylene (PTFE).

5. The method of claim 2, comprising determining, via the processor, the attenuation factor for the calibration block with a test sensor having known respective power for a respective first test sensor LED and a respective second test sensor LED.

6. The method of claim 1, wherein the first LED comprises a red LED and the second LED comprises an infrared (IR) LED.

7. The method of claim 1, comprising storing, via the processor, the power ratio in an erasable programmable read-only memory (EPROM) of the sensor.

8. The method of claim 1, comprising instructing, via the processor, a particular drive current to cause the first LED and the second LED to emit the light.

9. The method of claim 1, wherein the first photocurrent is indicative of a first amount of the light emitted by the first LED and that passes through the calibration structure, and wherein the second photocurrent is indicative of a second amount of the light emitted by the second LED that passes through the calibration structure.

10. A calibration system, comprising:
a sensor comprising:
a light emitter to emit a first light and a second light; and
a light detector to detect the first light and the second light; and
a calibration structure;
calibration processing circuitry; and
a calibration memory comprising instructions that, when executed by the calibration processing circuitry, cause the calibration processing circuitry to:
instruct a first light-emitting diode (LED) of the light emitter to emit the first light at a first wavelength and a second LED of the light emitter to emit the second light at a second wavelength;
receive a signal indicative of the first light at the first wavelength and the second light at the second wavelength;
determine a first power at the first wavelength and a second power at the second wavelength based on the signal; and
determine a power ratio as a ratio of the first power and the second power.

11. The calibration system of claim 10, wherein the sensor comprises a sensor memory, the sensor memory comprises an erasable programmable read-only memory (EPROM), and the instructions, when executed by the calibration processing circuitry, cause the calibration processing circuitry to transmit the power ratio to the EPROM.

12. The calibration system of claim 10, wherein the first LED comprises a red LED and the second LED comprises an infrared (IR) LED.

13. The calibration system of claim 10, wherein the instructions, when executed by the calibration processing circuitry, cause the calibration processing circuitry to provide a particular drive current to cause the first LED to emit the first light at the first wavelength and the second LED to emit the second light at the second wavelength.

14. The calibration system of claim 10, wherein the calibration structure comprises a calibration block with a known attenuation coefficient.

15. The calibration system of claim 10, wherein the calibration structure comprises a polymer material.

16. A method for utilizing storage on a sensor, the method comprising:
placing a first emitter of a sensor and a second emitter of the sensor at a calibration structure;
instructing the first emitter to emit light into the calibration structure at a first wavelength and the second emitter to emit light into the calibration structure at a second wavelength;
receiving a signal indicative of light detected at the calibration structure;
determining, based on the received signal, a first power at the first wavelength and a second power at the second wavelength;
determining a power ratio based on the first power and the second power;
transmitting the power ratio to the sensor for storage in a memory of the sensor.

17. The method of claim 16, wherein the calibration structure comprises an integrating sphere.

18. The method of claim 16, further comprising:
receiving one or more physiological signals from the sensor;
retrieving the power ratio from the memory; and
adjusting the one or more physiological signals based on the power ratio.

19. The method of claim 18, wherein adjusting comprises normalizing the one or more physiological signals based on the power ratio.

20. The method of claim 18, further comprising determining oxygen saturation based on the adjusted one or more physiological signals.
